(19) United States
(12) Patent Application Publication
(10) Pub. No.: US 2026/0146029 A1
(43) Pub. Date: May 28, 2026

(54) METHODS FOR THE TREATMENT OR PROPHYLAXIS OF ENDOMETRIOSIS

(71) Applicant: Gesynta Pharma AB, Stockholm (SE)

(72) Inventors: Patric Stenberg, Stockholm (SE); Charlotte Edenius, Stockholm (SE); Gunilla Ekstkröm, Stockholm (SE); Göran Tornling, Stockholm (SE); Eva Björkstrand, Stockholm (SE)

(21) Appl. No.: 19/107,831

(22) PCT Filed: Sep. 1, 2023
(86) PCT No.: PCT/EP2023/074032

§ 371 Date: Feb. 28, 2025

(30) Foreign Application Priority Data
Sep. 1, 2022 (GB) 2212749.2

(51) Int. Cl. C07D 235/14; A61K 31/4184; A61K 45/06

(52) U.S. Cl. CPC C07D 235/14; A61K 31/4184; A61K 45/06

(57) ABSTRACT

There is provided herein a compound of formula I, as defined herein, or a pharmaceutically acceptable salt thereof, and formulations comprising the same for use in the treatment or prophylaxis of endometriosis.

### CROSS-REFERENCE TO RELATED APPLICATIONS

This application is a 35 U.S.C. § 371 national stage filing of International Application No. PCT/EP2023/074032, filed on Sep. 1, 2023, which claims the benefit of priority to United Kingdom Patent Application No. 2212749.2, filed Sep. 1, 2022.

The entire contents of each of the above applications are incorporated herein by reference.

### FIELD OF THE INVENTION

The present invention relates to the use of a specific compound or pharmaceutically acceptable salt thereof, and pharmaceutical compositions, combinations and kits comprising the same, in the treatment or prophylaxis of endometriosis.

### BACKGROUND OF THE INVENTION

The listing or discussion of an apparently prior-published document in this specification should not necessarily be taken as an acknowledgement that the document is part of the state of the art or is common general knowledge.

Endometriosis is disorder in which endometrial tissue grow outside of the uterus, typically in the ovaries, fallopian tubes and around the lining of the pelvis, including around the bladder. In rare cases, such endometrial-like tissue may also be found in areas of the body which are remote from the reproductive organs.

With each menstrual cycle, this ectopic (extra-pelvic) endometrial-like tissue breaks down and becomes trapped, leading to considerable pain and discomfort. In the case of ovarian endometriosis, cysts called endometriomas may form. In many cases, the pain experienced is severe and detrimental to the patient's quality of life.

Over time, irritation caused by endometriosis may lead to the development of scar tissue and adhesions affecting organs in the pelvic region, which is often associated with symptoms such as chronic pelvic pain (cyclic and non-cyclic), painful periods, painful sex and pain on defecation and urination, and will commonly have a negative impact on fertility.

Although there is currently no cure for endometriosis, various treatments allow for management of the symptoms. Lesions, scar tissue and adhesions resulting from endometriosis may be removed through surgery, although this may be followed by relapse. There has also been considerable research into the use of hormonal contraceptive agents as prophylactic agents (see, for example, Zorbas, K. A. et al., Archives of Gynecology and Obstetrics, 292(1), 37-43 (2015)).

There remains, however, a need for an effective means for the long-term treatment and prophylaxis of endometriosis.

NSAIDs are amongst the world's most used and recognisable medications with billions of doses prescribed each year to treat inflammation, pain and fever. NSAIDs include traditional forms, such as ibuprofen, naproxen, indomethacin and diclofenac, as well as selective inhibitors of COX-2, such as celecoxib (Celebrex™) NSAIDs and COX-2 inhibitors reduce inflammation through inhibition of one or both isoforms of COX enzymes. The cyclooxygenase (COX) enzyme exists in two forms; one that is constitutively expressed in many cells and tissues (COX-1) and one that in most cells and tissues is induced by pro-inflammatory stimuli, such as cytokines, during an inflammatory response (COX-2).

COXs metabolise arachidonic acid to the unstable intermediate prostaglandin H₂ (PGH₂), which is further metabolized to other prostaglandins including PGE₂, PGF_{2α}, PGD₂, prostacyclin and thromboxane A₂. These arachidonic acid metabolites are known to have pronounced physiological and pathophysiological activity including pro-inflammatory effects. PGE2 in particular is known to be a strong pro-inflammatory mediator, and is also known to induce fever, inflammation and pain. Consequently, numerous drugs were developed with a view to inhibiting the formation of PGE2, predominantly by inhibition of COX-1 and/or COX-2.

Recently, concerns over cardiovascular side effects associated with the use of these drugs has resulted in a series of regulatory events including (i) the withdrawal of the blockbuster drug Vioxx in 2004, (ii) the introduction of ‘black box’ warnings on some NSAIDs from 2005 and on all drugs in this class since 2015, (iii) the withdrawal of Onsenal (celecoxib) for the prevention of cancer in 2011 and (iv) the reclassification of the over-the-counter medication diclofenac as prescription only in 2015 (UK). Now the fear of cardiovascular events caused by NSAIDs has become a public health issue resulting in the cautious prescribing of COX-2 selective drugs in favor of older style medication which are more toxic to the gut and a failure to realize the full clinical potential of NSAIDs in the prevention of cancer (Scarpignato, C. et al., BMC Med., 13, 55 (2015); Garcia Rodriguez, L. A., et al., Recent Results Cancer Res., 191, 67-93 (2013)). Moreover, the inhibition of COXs has the disadvantage that it results in the reduction of the formation of all metabolites downstream of PGH2, some of which are known to have beneficial properties.

Inhibition of mPGES-1 is a well-developed area of preclinical research with studies showing that its genetic deletion protects against inflammation, pain and cancer (Friesen, R. W. and Mancini, J. A., J Med Chem., 51, 4059-4067 (2008); Howe, L. R. et al., Prostaglandins Other Lipid Mediat., 106, 99-105 (2013); Korotkova, M. and Jakobsson P. J., Basic Clin Pharmacol Toxicol., 114, 64-69 (2014)). However, the lack of a complete understanding surrounding cardiovascular toxicity and other negative effects has meant that research and translation of mPGES-1 as a therapeutic target to replace COX-2 in the treatment of a wide range of diseases and disorders has been restricted.

WO 2012/022793 A1 describes the use of various mPGES-1 inhibitors in the treatment of a range of inflammatory disorders. WO 2017/144909 A1 describes the use of various mPGES-1 inhibitors in the treatment of diseases and disorders characterised by vasoconstriction. These disclosures do not teach or suggest the treatment or prophylaxis of endometriosis.

### DESCRIPTION OF THE INVENTION

We have now surprisingly found that a particular compound as described herein is an effective agent for the treatment or prophylaxis of endometriosis.

In a first aspect of the invention, there is provided a compound of formula I

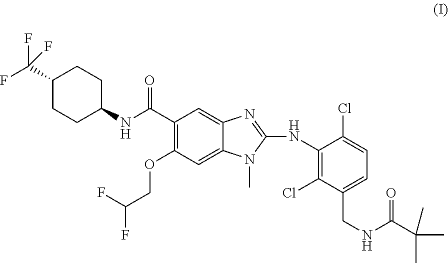 [SMILES: Cn1c(Nc2c(Cl)ccc(CNC(=O)C(C)(C)C)c2Cl)nc2cc(C(=O)N[C@H]3CC[C@H](C(F)(F)F)CC3)c(OCC(F)F)cc21]

or a pharmaceutically acceptable salt thereof, for use in the treatment or prophylaxis of endometriosis.

In an alternative first aspect of the invention, there is provided a method for the treatment or prophylaxis of endometriosis, comprising administering to a patient in need thereof a therapeutically effective amount of a compound of formula I as defined herein, or a pharmaceutically acceptable salt thereof.

In a further alternative first aspect of the invention, there is provided the use of a compound of formula I as defined herein, or a pharmaceutically acceptable salt thereof, in the manufacture of a medicament for the treatment or prophylaxis of endometriosis.

In a further alternative first aspect of the invention, there is provided the use of a compound of formula I as defined herein, or a pharmaceutically acceptable salt thereof, in the treatment or prophylaxis of endometriosis.

Unless indicated otherwise, all technical and scientific terms used herein have the same meaning as commonly understood by one of ordinary skill in the art to which this invention pertains.

The skilled person will understand that references herein to particular aspects of the invention will include references to all embodiments and particular features thereof.

Moreover, all embodiments of particular aspects of the invention may be combined with one or more other embodiments to form further embodiments without departing from the teaching of the invention.

### Compounds of the Invention

The skilled person will understand that references to a compound of formula I will refer to the following compound:

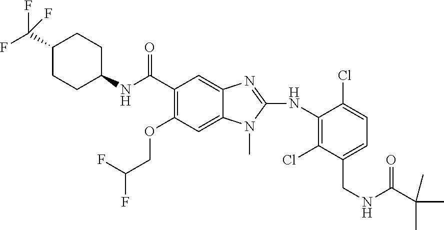 [SMILES: Cn1c(Nc2c(Cl)ccc(CNC(=O)C(C)(C)C)c2Cl)nc2cc(C(=O)N[C@H]3CC[C@H](C(F)(F)F)CC3)c(OCC(F)F)cc21]

which compound, together with pharmaceutically acceptable salts thereof, may also be referred to herein as the compound(s) of the invention, or the like.

The skilled person will also understand that the compound of formula I may be referred to by the chemical name 2-{2,6-dichloro-3-[(2,2-dimethyl-propionylamino)-methyl]-phenylamino}-6-(2,2-difluoroethoxy)-1-methyl-1H-benzoimidazole-5-carboxylic acid (trans-4-trifluoromethylcyclohexyl)-amide.

Pharmaceutically-acceptable salts include acid addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxy-benzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxyethanesulphonate, 1- or 2-naphthalene-sulphonate or 1,5-naphthalenedisulphonate salts) or sulphate (e.g. dihydrogen sulphate), pyrosulphate, bisulphate, sulphite, bisulphite, phosphate, monohydrogenphosphate, dihydrogenphosphate, metaphosphate, pyrophosphate or nitrate salts, and the like.

Particular pharmaceutically acceptable salts that may be mentioned include dihydrogen sulphate (also referred to as hydrogen sulphate; H₂SO₄) salts.

For the avoidance of doubt, the skilled person will understand that the compounds of the invention may exist as solids, and thus the scope of the invention includes all amorphous, crystalline and part crystalline forms thereof, and may also exist as oils.

Where such compounds exist in crystalline and part crystalline forms, such forms may include solvates, which are included in the scope of the invention. Such compounds may also exist in solution.

Compounds of the invention may also exhibit tautomerism. All tautomeric forms and mixtures thereof are included within the scope of the invention.

As described herein, compounds of the invention have a specific orientation of substituents. For the avoidance of doubt, compounds of the invention have the stereochemistry as depicted, which may be referred to as trans orientation.

The skilled person will understand that the present invention may utilise not only compounds of the invention as such, but also compounds which may be administered to a patient (e.g. parenterally or orally) and thereafter be metabolised in the body to form compounds of the invention. Such compounds may be described as prodrugs of compounds of the invention.

### Medical Uses

As described herein, compounds of the invention are of use in the treatment or prophylaxis of endometriosis in patients in need thereof.

The skilled person will understand that references to particular diseases and disorders will take their normal meaning as understood by those skilled in the art.

Similarly, the skilled person will understand that references to the treatment of a particular disease (or, similarly, to treating that disease) take their normal meaning in the field of medicine. In particular, the term may refer to achieving a reduction in the severity of one or more clinical symptom associated with the disease, even if diagnosis has yet to be determined at the time of treatment of such symptoms. Such reductions may be measured using objective analysis (e.g. the measurement of physiological factors and/or the visualisation of disease markers, such as a reduction on the size and/or number of lesions) and/or through subjective analysis (for example, by assessment through examination by a physician, or through patient reported outcomes, such as reduction of pain, e.g. as assessed using appropriate pain scales, and improved assessment of quality of life).

The skilled person will understand that references to the prophylaxis of a particular disease take their normal meaning in the field of medicine. In particular, the term may refer to achieving a reduction in the likelihood of the patient (or healthy subject) developing the disease (for example, at least a 10% reduction, such as at least a 20%, 30% or 40% reduction, e.g. at least a 50% reduction). The skilled person will also understand that prophylaxis of a particular disease may also be referred to as prevention of that disease, and that these terms may be used interchangeably.

The skilled person will understand that prophylaxis may be performed in a patient not suffering from the relevant disorder (i.e. such that the relevant disorder does develop). In particular, prophylaxis may be performed in a patient not suffering from the relevant disorder but at risk of developing (e.g. due to their having previously had) the relevant disorder.

In certain embodiments, references to the treatment or prophylaxis of endometriosis may refer to a reduction (i.e. a clinically significant reduction) of or reduction in the formation of lesions, adhesions and/or scar tissue associated with endometriosis.

The skilled person will be able to identify patients (who may, in such instances, be otherwise healthy) for whom prophylaxis of a disease or disorder will be required, such as those at risk of developing the relevant disorder (e.g. patients who have previously been treated for that disorder but do not at that time have the disorder).

The skilled person will be able to perform the determination of whether a patient has the relevant disorder or is at risk of developing the relevant disorder using techniques that are routine in that art. For example, patients not having the relevant disorder may be patients who are not experiencing associated symptoms (such as pain or infertility resulting from the disease) at the time that said prophylaxis is performed.

Without wishing to be bound by theory, it is believed that compounds of the invention may allow for the treatment of endometriosis in a manner that provides therapeutic benefits additional to those provided by similar medications prescribed for the treatment of the disease, such as those prescribed for the treatment of pain (e.g. non-steroidal anti-inflammatory drugs (NSAIDs), and paracetamol). Moreover, it is believed that compounds of the invention may be administered in a continuous manner with a lower risk of adverse drug reaction than would be associated with similar medications prescribed for the treatment of the disease, such as those prescribed for the treatment of pain and/or hormonal agents.

In a particular embodiment, the treatment or prophylaxis (e.g. treatment) as described herein requires continuous daily administration (i.e. daily administration of a therapeutically effective amount as described herein) of the compound of the invention.

In more particular such embodiments, the treatment or prophylaxis is for an extended period, which may be referred to as long term treatment (which may refer to a period of at least six weeks, such as a period of at least 12 weeks, at least 24 weeks or at least 48 weeks).

In more particular such embodiments, the continuous daily administration (including continuous daily administration for an extended period, i.e. long term continuous daily administration) will include periods of time when the patient is not (a) experiencing significant pain (which may be defined as greater than moderate pain) and/or (b) menstruating.

As used herein, references to patients will refer to a living subject being treated, including mammalian patients. In particular embodiments, references to patients will refer to human patients.

As such, in a particular embodiment the treatment or prophylaxis as described herein is performed in a mammal (e.g. a human).

The skilled person will understand that endometriosis is a disease of the female reproductive system and, as such, patients will be expected to be biologically female patients, typically of reproductive age.

As used herein, the term therapeutically effective amount (which may also be referred to as an effective amount) will refer to an amount (i.e. a dose) of a compound that confers a therapeutic effect on the treated patient. The effect may be objective (i.e. measurable by some test or marker, such as by achieving a particular level of inhibition of a target enzyme, or achieving a particular effect on lesion size and/or number as assessed by imaging techniques) or subjective (i.e. the subject gives an indication of and/or feels an effect). The skilled person will be able to determine a suitable therapeutically effective dose using techniques that are routine in the art.

For example, particular doses that may be mentioned include those in the range of from about 10 to about 200 mg per day (of the non-salt, free base form; adjusted if used in the form of a salt), which may be administered (e.g. orally, such as in the form of a tablet or capsule) as a single daily dose (once daily) or divided between two daily doses (twice daily).

The skilled person will understand that both the treatment and prophylaxis of diseases as described herein may be achieved through repeated administration of the compounds of invention, such as may be achieved by daily (e.g. once or twice in a 24 hour period) administration of a suitable dose and/or administration in a form that allows for extended release of the active ingredient (e.g. release over a period of at least 12 hours, such as at least 24 hours) from a suitable dosage form as known to those skilled in the art.

The skilled person will understand that prophylaxis of a particular disease or disorder may be performed in combination with the treatment of another disease or disorder, or vice versa, with the disease being treated being either unrelated to or an underlying (e.g. causative) factor of the disease or disorder the prophylaxis of which is provided.

Further, the skilled person will understand that the present invention may be particularly suited to the treatment of chronic diseases, such as chronic manifestations of those diseases and disorders mentioned herein. As used herein, the skilled person will understand that references to chronic diseases will refer to diseases that persist for an extended period of time (e.g. for at least three months, such as at least six months or at least one year).

Without wishing to be bound by theory, it is believed that compounds of the invention may be of particular use in treating endometriosis in patients who have not tolerated, have switched from, are unable to be prescribed or are refractory to treatment with other therapeutic agents for the treatment or prophylaxis (e.g. treatment) of the same disease or disorder (such as NSAIDs, hormonal agents, paracetamol, anticonvulsants, TCAs, SSRI/SNRIs and/or opioids).

In a particular embodiment, the treatment or prophylaxis (e.g. treatment) is in a patient who:
(i) has not tolerated;
(ii) has switched from or reduced the dose of;
(iii) is unable to be prescribed; or
(iv) is refractory to,

one or more other therapeutic agents for the treatment or prophylaxis (e.g. treatment) of the same disease.

The skilled person will understand that the reference to one or more other therapeutic agents for the treatment or prophylaxis (e.g. treatment) of the same disease will refer to one or more (e.g. one) other therapeutic agent(s) previously prescribed for (and administered to) the patient for said purpose, which may be referred to as one or more previous medications, which are other than compounds of the invention.

Other therapeutic agents for the treatment or prophylaxis (e.g. treatment) of endometriosis as described herein (including all embodiments and particular features thereof) include NSAIDs, hormonal agents, paracetamol, anticonvulsants, TCAs, SSRI/SNRIs and/or opioids.

In a particular embodiment, the one or more other therapeutic agents for the treatment or prophylaxis (e.g. treatment) of endometriosis is an NSAID.

Particular NSAIDs that may be mentioned include aspirin, celecoxib, ibuprofen, naproxen and diclofenac, indomethacin and etoricoxib.

In a particular embodiment, the one or more other therapeutic agents for the treatment or prophylaxis (e.g. treatment) of endometriosis is a CNS acting agent.

Particular CNS acting agents that may be mentioned include TCA (e.g. amitriptylin), anticonvulsants (e.g. gabapentin, pregabalin), SSRI/SNRI (e.g. duloxetine, milnacipran) and opioids (e.g. morphine, buprenorphine).

In a particular embodiment, the one or more other therapeutic agents for the treatment or prophylaxis (e.g. treatment) of endometriosis is a hormonal agent.

Particular hormonal agents that may be mentioned include hormone contraceptives, such as the combined (oestrogen and progesterone) contraceptive pill, progestogens, antiprogestogens, the contraceptive patch and gonadotrophin-releasing hormone (GnRH) analogues (agonists and antagonists; which may be administered with low doses of oestrogen), modulators of hormone synthesis or metabolism (such as aromatase inhibitors, 17β-hydroxysteroid dehydrogenase inhibitors and steroid sulfatase inhibitors).

In a particular embodiment, the one or more other therapeutic agents for the treatment or prophylaxis (e.g. treatment) of endometriosis is paracetamol.

In an alternative embodiment, the one or more other therapeutic agents for the treatment or prophylaxis (e.g. treatment) of endometriosis is other than paracetamol.

For example, in certain embodiments where one or more other therapeutic agents for the treatment or prophylaxis (e.g. treatment) of endometriosis is other than paracetamol, treatment or prophylaxis with paracetamol may be continued together with treatment or prophylaxis with compounds of the invention.

In a particular embodiment, the treatment or prophylaxis (e.g. treatment) is in a patient who has not tolerated one or more other therapeutic agents for the treatment or prophylaxis (e.g. treatment) of the same disease.

The skilled person will understand that references to a patient who has not tolerated one or more other therapeutic agents for the treatment or prophylaxis (e.g. treatment) of the same disease will refer to patients for whom prior such treatment or prophylaxis has been deemed to be unsuitable by a physician or suitable medical professional based on suitable medical parameters, such as the level of adverse events experienced by the patient and/or a lack of willingness of the patient to continue with such treatment or prophylaxis.

In particular embodiments, such as wherein the one or more other therapeutic agents for the treatment or prophylaxis (e.g. treatment) of the same disease are hormonal agents, such adverse events may be selected from:
weight gain;
decreased libido;
osteoporosis;
suicidal ideation;
depression and/or anxiety;
low mood; and
symptoms of menopause (such as hot flushes/hot flashes).

In particular embodiments, such as wherein the one or more other therapeutic agents for the treatment or prophylaxis (e.g. treatment) of the same disease are NSAIDs, such adverse events may be selected from cardiovascular adverse events (including myocardial infarction (MI), stroke, heart failure (HF), blood pressure elevation, atrial fibrillation and venous thromboembolism);
renal impairment; and
asthma;
gastro intestinal adverse events (including dyspepsia, bleeding, ulceration, and perforation of the stomach or intestines).

In particular embodiments, such as wherein the one or more other therapeutic agents for the treatment or prophylaxis (e.g. treatment) of the same disease are CNS active agents, such adverse events may be selected from:
anticholinergic effects;
mood disorders;
drowsiness;
blurred vision;
dependence;
constipation; and
tolerance development.

In a further embodiment, the treatment or prophylaxis (e.g. treatment) is in a patient who has switched from or reduced the dose of one or more other therapeutic agents for the treatment or prophylaxis (e.g. treatment) of the same disease.

The skilled person will understand that references to a patient who has switched from one or more other therapeutic agents for the treatment or prophylaxis (e.g. treatment) of the same disease will refer to patients who have ceased treatment or prophylaxis with the one or more other therapeutic agents and have switched to treatment or prophylaxis with (e.g. have begun treatment or prophylaxis (e.g. treatment) with immediately thereafter, which may refer to the absence of intervening treatment or prophylaxis (e.g. treatment) of the same disease) a compound of the invention, which may be as directed by a physician or suitable medical professional.

The skilled person will understand that references to a patient who reduced the dose of one or more other therapeutic agents for the treatment or prophylaxis (e.g. treatment) of the same disease will refer to patients who continue treatment or prophylaxis with the one or more other therapeutic agents but, prior to beginning treatment or prophylaxis with a compound of the invention, have reduced the dose of one or more other therapeutic agents, which may be as directed by a physician or suitable medical professional.

The skilled person will also understand that references to reducing the dose of one or more other therapeutic agents will refer to the patient receiving a lower daily amount thereof, such as by reducing the dose administered at each occurrence and/or reducing the frequency of administration during a day, and/or reducing the frequency of treatment, such as by reducing the number of days on which treatment is administered (e.g. during a typical 30 day period).

For the avoidance of doubt, references to reducing the dose of one or more other therapeutic agents will indicate that the treatment or prophylaxis with one or more other therapeutic agents is continued (at a reduced level) during treatment or prophylaxis with compounds of the invention.

In a particular embodiment, the treatment or prophylaxis (e.g. treatment) is in a patient who has switched from one or more other therapeutic agents for the treatment or prophylaxis (e.g. treatment) of the same disease.

In a further embodiment, the treatment or prophylaxis (e.g. treatment) is in a patient who has reduced the dose of one or more other therapeutic agents for the treatment or prophylaxis (e.g. treatment) of the same disease.

In a further embodiment, the treatment or prophylaxis (e.g. treatment) is in a patient who is unable to be prescribed one or more other therapeutic agents for the treatment or prophylaxis (e.g. treatment) of the same disease.

The skilled person will understand that references to a patient who is unable to be prescribed one or more other therapeutic agents for the treatment or prophylaxis (e.g. treatment) of the same disease will refer to patients who cannot be prescribed one or more other therapeutic agents as determined by a physician or suitable medical professional based on suitable medical parameters, such as the health, physiology and/or medical history patient, or other similar factors, and/or a lack of willingness of the patient to accept such one or more other therapeutic agents.

In a particular embodiment, the treatment or prophylaxis is in a patient who cannot be prescribed one or both of NSAIDs and hormonal agents, such as those described herein.

In a particular embodiment, the treatment or prophylaxis is in a patient who cannot be prescribed NSAIDs, such as those described herein.

In a particular embodiment, the treatment or prophylaxis is in a patient who cannot be prescribed hormonal agents, such as those described herein.

In a particular embodiment, the patient cannot be prescribed the one or more other therapeutic agents based on suitable medical parameters, such as the health, physiology and/or medical history of the patient.

In particular embodiments, the treatment or prophylaxis as described herein is in a patient who cannot be prescribed hormonal agents (such as those described herein) due to the patient having previously experienced (i.e. having a history of) one or more condition selected from:
depression and/or anxiety;
low mood; and
symptoms of menopause (such as hot flushes/hot flashes).

In particular embodiments, the treatment or prophylaxis as described herein is in a patient who cannot be prescribed the one or more other therapeutic agents due to the patient trying to conceive, i.e. intending to become pregnant (including patients undergoing fertility treatment, such as in vitro fertilisation (IVF)).

For example, in more particular embodiments, the treatment or prophylaxis as described herein is in a patient who cannot be prescribed NSAIDs and/or hormonal agents (such as those described herein) due to the patient trying to conceive.

In particular such embodiments, where the patient is trying to conceive, the one or more other therapeutic agents may be other than paracetamol (such as those described herein other than paracetamol).

In particular embodiments, the treatment or prophylaxis as described herein is in a patient who cannot be prescribed NSAIDs (such as those described herein) due to the patient having previously experienced (i.e. having a history of) one or more of: cardiovascular adverse events (including myocardial infarction (MI), stroke, heart failure (HF), blood pressure elevation, atrial fibrillation and venous thromboembolism); gastro intestinal adverse events (including stomach ulcers).

In a particular embodiment, the patient cannot be prescribed the one or more other therapeutic agents due to a lack of willingness (e.g. refusal) of the patient to accept such one or more other therapeutic agents.

In particular embodiments, the lack of willingness of the patient to accept such one or more other therapeutic agents is a result of (i.e. is caused by) their cultural, societal, religious and/or political practices or beliefs.

For example, in a particular embodiment, the patient cannot be prescribed hormonal agents due to a lack of willingness of the patient to accept such therapeutic agents due to cultural, societal, religious and/or political practices or beliefs.

In a further embodiment, the treatment or prophylaxis (e.g. treatment) is in a patient who is refractory to one or more other therapeutic agents for the treatment or prophylaxis (e.g. treatment) of the same disease.

The skilled person will understand that references to a patient who is refractory to one or more other therapeutic agents for the treatment or prophylaxis (e.g. treatment) of the same disease will refer to patients for whom such treatment or prophylaxis has been deemed to be unsuitable, such as by a physician or suitable medical professional, based on a lack of efficacy, which may refer to the disease not responding (or not responding adequately) to such treatment or prophylaxis.

In a particular embodiment, the treatment or prophylaxis (e.g. treatment) is in a patient who is refractory to treatment or prophylaxis (e.g. treatment) of the same disease with NSAIDs and/or hormonal agents.

In a particular embodiment, the treatment or prophylaxis (e.g. treatment) is in a patient who is refractory to treatment or prophylaxis (e.g. treatment) of the same disease with NSAIDs.

In a particular embodiment, the treatment or prophylaxis (e.g. treatment) is in a patient who is refractory to treatment or prophylaxis (e.g. treatment) of the same disease with hormonal agents.

As described herein, the treatment or prophylaxis with compounds of the invention may allow for treatment or prophylaxis in, or be particularly beneficial for, certain patient groups.

In a particular embodiment, the treatment or prophylaxis (e.g. treatment) is in patients who are experiencing and/or have previously experienced:
depression and/or anxiety;
low mood; and/or
symptoms of menopause (such as hot flushes/hot flashes).

In a particular embodiment, the treatment or prophylaxis (e.g. treatment) is in patients who are experiencing, have an increased risk of experiencing and/or have previously experienced:
cardiovascular adverse events (including myocardial infarction (MI), stroke, heart failure (HF), blood pressure elevation, atrial fibrillation and venous thromboembolism); renal impairment; and/or
gastro intestinal adverse events (including stomach ulcers).

In a particular embodiment, the treatment or prophylaxis (e.g. treatment) is in a patient who is trying to conceive, i.e. intending to become pregnant (including patients undergoing fertility treatment, such as in vitro fertilisation (IVF)).

In a particular embodiment, the treatment or prophylaxis of the relevant disease may further comprise treatment or prophylaxis of reduced fertility (e.g. infertility), which may refer to reduced fertility resulting from (i.e. caused by the effects of) the disease.

In such embodiments, the treatment or prophylaxis of reduced fertility may result in reduced use of IVF and increased number of pregnancies (i.e. the take home baby rate).

As such, compounds of the invention may also be useful in prophylaxis or treatment (e.g. prophylaxis) of infertility associated with endometriosis, which may result in reduced use of IVF and/or increased number of pregnancies (take home baby rate).

Thus, in an alternative aspect of the invention, there is provided a compound of formula I as defined herein, or a pharmaceutically acceptable salt thereof, for use in the prophylaxis or treatment of infertility associated with endometriosis.

In a further alternative aspect of the invention, there is provided a method for the prophylaxis or treatment of infertility associated with endometriosis, comprising administering to a patient in need thereof a therapeutically effective amount of a compound of formula I as defined in Claim 1, or a pharmaceutically acceptable salt thereof.

In a further alternative aspect of the invention, there is provided the use of a compound of formula I as defined herein, or a pharmaceutically acceptable salt thereof, in the manufacture of a medicament for the prophylaxis or treatment of infertility associated with endometriosis.

The skilled person will understand that references to infertility associated with endometriosis may refer to reduced or impaired fertility (ability to conceive) in patients with endometriosis, in which the infertility has been determined to or is expected to result from the effects of the endometriosis (such as the formation of lesions, adhesions and/or scar tissue affecting the reproductive organs, e.g. around the uterus and/or ovaries).

In a particular embodiment, the treatment may be in a patient who is experiencing significant (which may be defined as moderate or greater) pain, which pain may occur during menstruation or between menstrual cycles (e.g. between menstrual cycles).

In such embodiments, the treatment may result in treatment of such pain.

In particular embodiments, the treatment or prophylaxis (e.g. treatment) may be in a patient who is experiencing pain associated with:
poor quality of life;
impaired capacity to perform daily life activities;
cognitive dysfunction;
poor social interaction;
reduced libido;
dyspareunia;
symptoms of depression and anxiety; and/or
impaired (or reduced) mobility.

In particular embodiments, the treatment or prophylaxis (e.g. treatment) may be in a patient who is experiencing chronic pain, such as chronic pain associated with: dysmenorrhea (before, during or after menstruation);
lower back pain;
ovulatory associated pain;
dyspareunia;
muscle and/or joint pain;
shortness of breath, chest pain and/or cough, and/or in certain instances, collapsed lung;
painful defecation;
chronic pelvic pain;
cyclical pelvic pain;
dysuria; and/or
burning vaginal pain.

In particular embodiments, the treatment or prophylaxis (e.g. treatment) may be in a patient who is experiencing chronic pelvic pain.

In such embodiments, the treatment may result in treatment of such associated factors.

In particular embodiments, the treatment may be in a patient who is experiencing irregular, reduced or inhibited menstrual cycles.

In such embodiments, the treatment may result in the patient achieving regular menstrual cycles.

In a particular embodiment, the treatment or prophylaxis (e.g. treatment) is in a patient who is not suitable for surgical treatment for the treatment of relevant disease, as determined by a physician or suitable medical professional based on suitable medical parameters.

In a particular embodiment, the treatment or prophylaxis (e.g. treatment) is in a patient who has previously received surgical treatment for the treatment of the relevant disease, which may be referred to as post-operative treatment or prophylaxis.

In a particular embodiment, the post-operative treatment is in patients experiencing significant pain (which may be defined as greater than moderate pain).

As described herein, the treatment or prophylaxis (e.g. treatment) with compounds of the invention may be associated with effects other the treatment or prophylaxis of pain. For example, the treatment or prophylaxis (e.g. treatment) with compounds of the invention may be associated with (e.g. result in/induce) a reduction in the size and/or number of endometroid lesions (which may also be referred to as foci and/or cysts), such as endometrioma.

### Pharmaceutical Formulations, Combinations and Kits

The skilled person will understand that compounds of the invention may be administered in the form of pharmaceutical formulations.

In a second aspect of the invention, there is provided a pharmaceutical formulation comprising a compound of formula I as defined herein, or a pharmaceutically acceptable salt thereof, and optionally one or more pharmaceutically acceptable excipients for use in the treatment or prophylaxis of endometriosis.

In an alternative second aspect of the invention, there is provided a method for the treatment or prophylaxis of endometriosis comprising administering to a patient in need thereof a therapeutically effective amount of a pharmaceutical formulation comprising a compound of formula I as defined herein, or a pharmaceutically acceptable salt thereof, and optionally one or more pharmaceutically acceptable excipients.

The skilled person will understand that compounds of the invention, and pharmaceutical formulations comprising the same, may be administered systemically and/or locally (e.g. topically).

The skilled person will understand that compounds of the invention, and pharmaceutical formulations comprising the same, will normally be administered orally, intravenously, subcutaneously, buccally, rectally, intravaginally, dermally, nasally, bronchially, sublingually, intranasally, topically, intrauterine, intraperitoneally, by any other parenteral route or via inhalation, in a pharmaceutically acceptable dosage form.

In particular, compounds that are mPGES-1 inhibitors may be administered by way of known pharmaceutical formulations (i.e. compositions suitable for use in medicine), including tablets, capsules or elixirs for oral administration, suppositories for rectal administration, pessaries for intravaginal administration, sterile solutions or suspensions for parenteral or intramuscular administration, and the like.

As used herein, the skilled person will understand that references to a pharmaceutically acceptable excipient will include references to pharmaceutically acceptable adjuvants, diluents and/or carriers, as known to those skilled in the art. For example, suitable pharmaceutically acceptable excipients and methods of formulation may include those described in “Handbook of Pharmaceutical Excipients” by P. J. Sheskey, B. C. Hancock, G. P. Moss and D. J. Goldfarb, Ninth edition, Pergamon Press, 1995.

The skilled person will understand that mPGES-1 inhibitors as described herein, and pharmaceutical formulations comprising the same, may act systemically and/or locally (i.e. at a particular site).

Thus, pharmaceutical formulations as described herein may be administered orally, intravenously, subcutaneously, buccally, rectally, dermally, nasally, bronchially, by any other parenteral route or via inhalation, in a pharmaceutically acceptable dosage form. Alternatively, particularly the mPGES-1 inhibitor is intended to act locally, compounds of the invention may be administered topically (in which case the pharmaceutical formulation may be a formulation for topical administration).

Depending on, for example, the potency and physical characteristics of compounds of the invention, pharmaceutical formulations that may be mentioned include those in which the compound is present in at least 1% (or at least 10%, at least 30% or at least 50%) by weight; that is, wherein the ratio of active ingredient to the other components (i.e. the excipients) of the pharmaceutical formulation is at least 1:99 (or at least 10:90, at least 30:70 or at least 50:50) by weight.

In certain embodiments, treatment or prophylaxis using compounds of the invention as described herein may also be combined with one or more other (i.e. different) therapeutic agents that are useful in the treatment or prophylaxis of the same disease (i.e. endometriosis).

Thus, in a particular embodiment, the pharmaceutical formulation further comprises one or more additional therapeutic agent (i.e. other than the compound of formula I or pharmaceutically acceptable salt thereof) for the treatment or prophylaxis of endometriosis.

The skilled person will understand that pharmaceutical formulations comprising additional therapeutic agents may be presented in the form of a single formulation (i.e. a formulation comprising all of the relevant therapeutic agents) or as combination products that provide for the administration of the compounds of the invention in conjunction with the one or more additional therapeutic agent as separate formulations (i.e. distinct formulations wherein at least one of those formulations comprises the compound of the invention and at least one comprises the other (additional) therapeutic agent).

In a third aspect of the invention, there is provided a combination product comprising:
(I) a compound of formula I as defined in the first aspect of the invention, or a pharmaceutically acceptable salt thereof; and
(II) one or more other therapeutic agent that is useful in the treatment or prophylaxis of endometriosis,

wherein each of components (I) and (II) is formulated in admixture, optionally with one or more a pharmaceutically-acceptable excipient.

In a fourth aspect of the invention, there is provided a kit-of-parts comprising:
(a) a pharmaceutical formulation as defined in the second aspect of the invention; and
(b) a pharmaceutical formulation comprising one or more other therapeutic agent that is useful in the treatment or prophylaxis of endometriosis, optionally in admixture with one or more pharmaceutically-acceptable excipients,

wherein components (a) and (b) are each provided in a form that is suitable for administration in conjunction with the other.

The skilled person will be able to identify suitable compounds for the treatment or prophylaxis of endometriosis as known in the art. For example, compounds for the treatment or prophylaxis of endometriosis may include paracetamol, non-steroidal anti-inflammatories (NSAIDs), hydroxysteroid dehydrogenase inhibitors, androgen agonists (e.g. danazol), prolactin antagonists, dopamine agonists, P2X2/P2X3/P2X4 antagonists, modulators of gonadotropin releasing hormone (GnRH), hormonal contraceptive agents and modulators of hormone synthesis or metabolism (such as aromatase inhibitors, 17p-hydroxysteroid dehydrogenase inhibitors and steroid sulfatase inhibitors).

Particular compounds for the treatment or prophylaxis of endometriosis will include paracetamol.

The skilled person will also understand that treatment or prophylaxis with compounds of the invention as described herein may also be combined with medical procedures performed for the treatment or prophylaxis of the same disease, such as surgical procedures. For example, the use of compounds of the invention may be used post-operative (i.e. following surgical treatment for the diseases as described herein).

### Preparation of Compounds

Compounds of the invention are known in the literature and may be commercially available. Such compounds may be prepared by conventional synthetic procedures, in accordance with standard techniques, from available starting materials using appropriate reagents and reaction conditions. In this respect, the skilled person may refer to standard reference materials such as: “Comprehensive Organic Synthesis” by B. M. Trost and I. Fleming, Pergamon Press, 1991; “Heterocyclic Chemistry” by J. A. Joule, K. Mills and G. F. Smith, 3^{rd} edition, published by Chapman & Hall; and “Comprehensive Heterocyclic Chemistry II” by A. R. Katritzky, C. W. Rees and E. F. V. Scriven, Pergamon Press, 1996.

In particular, the skilled person may refer to the synthetic methods referred to in WO 2012/022793 A1 (in particular, the examples provided therein, such as Example 1, and the associated data), the contents of which are incorporated herein by reference.

Stereoisomers may be obtained by separation using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired stereoisomers may be made by reaction of starting materials with appropriate stereochemistry under conditions which will not cause racemisation or epimerisation, or by reaction with an appropriate chiral reagent or chiral catalyst, all under conditions known to the skilled person.

As described herein, the present invention is based on the unexpected discovery that compounds of the invention are surprisingly effective in the treatment and prophylaxis of endometriosis.

As such, compounds of the invention may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, compounds of the invention may have the advantage that they are more efficacious and/or exhibit advantageous properties in vivo.

Without wishing to be bound by theory, it is thought that compounds of the invention allow for an effective means for the treatment or prophylaxis of endometriosis by reducing endometriosis lesion size and or number thereof, which in turn allows for improved effects in reducing pain, inflammation, fibrosis and/or adhesions associated with the disease and thus improving, inter alia, the behaviour and well-being of the patient. The lesion load may mechanically cause pain through number, size and localization, for example lesions, adhesions and scar tissue in the uterus/ovarian ligaments may contribute to dyspareunia, and therefore reduction in lesion load has beneficial effects in addressing these symptoms. It is thought that such effects are achieved together with a direct effect on pain and inflammation, further contributing to the therapeutic advantages. These unexpected properties allow for a more effective treatment, render the compounds suitable for use as prophylactic agents, and allow for treatment and prophylaxis in particular patient groups. The use of compounds of the invention may also allow for the prophylaxis or treatment of infertility associated with endometriosis, which may result in reduced use of IVF and increased number of pregnancies (take home baby rate).

### SUMMARY OF THE FIGURES

FIG. 1 shows the design of the study as reported in the examples.

FIG. 2 shows the changes in blood flow observed over the course of the study described in the examples.

FIG. 3 shows the changes in individual number of foci observed over the course of the study described in the examples.

FIG. 4 shows the changes in the number of endometriotic foci over the course of the study described in the examples.

FIG. 5 shows the changes in feeding tree behaviour over the course of the study.

FIG. 6 shows the changes in WGTA behaviour over the course of the study.

FIG. 7 shows the changes in grooming behaviour over the course of the study.

### EXAMPLES

The present invention is illustrated by way of the following examples, which are not intended to be limiting on the scope of the invention.

### Materials

### Compounds

The substance as depicted below (prepared according to the procedure in WO 2012/022793 A1) was orally administered (as the hydrogen sulphate salt thereof) for 6 weeks to common marmoset monkeys at a dose of 6.67 mg/kg twice/day (calculated based on the free base, with the actual weight of compound administered calculated as required for the hydrogen sulphate salt).

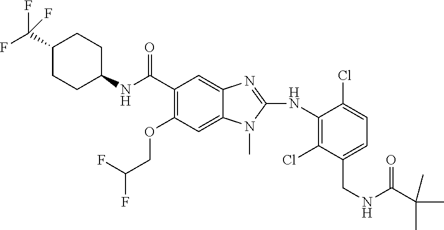 [SMILES: Cn1c(Nc2c(Cl)ccc(CNC(=O)C(C)(C)C)c2Cl)nc2cc(C(=O)N[C@H]3CC[C@H](C(F)(F)F)CC3)c(OCC(F)F)cc21]

Characterization of test animals

| | |
|---|---|
| Species, strain | Marmoset monkeys (Callithrix jacchus) |
| Sex | Female |
| Number | 8 |
| Status | Established endometriosis |

The age of the marmosets ranged between 3 and 16 years. All animals were female and had established endometriosis over years. Induction of endometriosis was performed as described in Einspanier et al., Mol Human Reprod., 12(5), 291-9 (2006). Some animals were induced with endometriosis several years ago and some newly induced.

### Preparation of Dosing Solutions

The compound was formulated weekly according to this protocol. The compound was dosed as a suspension made in 0.2% Natrosol© 250 HX (hydroxyethyl cellulose) in demineralized water and the dosing volume was 200 μl/kg. The suspension was stored securely covered until administration, protected from light, and refrigerated (2 to 8° C.).

### Animal Care

### Housing

The animals were pair caged with male marmoset monkey under standard conditions with a 12-h light and darkness cycle. The room temperature (24° C.) and humidity (50%) was kept constant. Cleaning of the room was carried out as described in the standard procedure of the animal facility.

### Food

Animals were fed two times per day. Morning pap was given between 7.00 and 8.00 h in the morning. The morning pap contained baby fruit pap, bananas, vitamin and mineral mixture. The lunch food was present around midday and contained fruits, high proteins, carbohydrates and vegetables. Marmosets pellets (Sniff) were available the whole day.

### Water

The animals had access to tap water ad libitum. The water in macrolon bottles was changed daily.

### Ethics

The study protocol was approved by the local ethical committee for animal experiments.

### Experimental Design

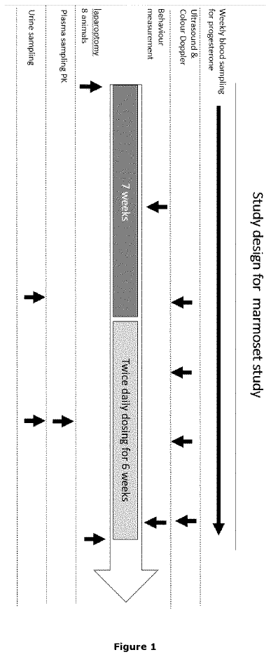

The study design is presented in FIG. 1.

### General Health Status

All animals were weighed weekly before and during the treatment. Health body checks including clinical signs as well as blood chemistry analysis were carried out in a regular manner by the responsible vet.

### Urine and Blood Sampling for Progesterone Analysis

For each animal urine samples for analysis of prostaglandin profile were collected before and once during treatment. The urine samples were stored at −80° C. until transport. For each animal blood samples were collected, one before and one during treatment, for measurement of plasma concentration.

For analysis of serum progesterone concentration to monitor the reproductive cycle, blood was collected weekly before and during treatment. Serum was separated from blood samples by centrifugation (2600 rpm 10 min, Eppendorf Centrifuge 5810 R) within two hours of sampling and stored at minus 80° C.

### Colour Doppler Ultrasonic Scanning

Colour Doppler ultrasonic scanning was done before and 3 times during the treatment period to follow blood flow. Colour doppler scanning was carried out as described by Einspanier (ibid).

### Laparotomy

Laparotomy was carried out twice, seven weeks before treatment start of treatment and immediately after end of treatment. There was a duration of at least 3 months between each laparotomy.

After pre-medication with Göttinger Mischung M II (Ketamin, Xylazin and Atropin mixture), an inhalation anaesthesia with Isofluran was carried out which last in average 60 minutes. The abdomen was opened beside line alba for 1 cm range to visualise the reproductive tract and bladder. The endometriotic lesions were characterized and documented in pictures. During the second laparoptomy samples from the lesions were collected. Postoperative care of the animals was carried out as required by local law. The collected tissue samples were fixed in paraformaldehyde and embedded in paraffin or stored at −80° C. Before abdominal suture, all organs were replaced in their place. All females were separated from their males for some hours due to medical reasons and obtained antibiotics for some days.

### Behaviour Analysis

Different behavioural test systems were used to study pain and discomfort, social comfort, memory, learning and activity were set up. All primates used in the study were familiar with the test-systems (for a period of more than 6 months) to confirm that the obtained results are related to the medication and not a learning effect over time.

Two test systems were used in the study as described in Arnold et al., J Med Primatol., 40, 317-326 (2011).
1. Feeding tree
2. Wisconsin General Testing apparatus (WGTA) Before and during the study documentation of social interaction, such as grooming, were performed.

### Statistical Method

As the data were not normally distributed, all analyses were performed by Wilcoxon Signed Rank Test. Results

### Clinical Signs, Viability and Observation of the Animals

There were no changes in clinical signs, general health behaviour of the study animals during the study period. All blood laboratory parameters were in the physiological range before and after treatment.

### Body Weight

There was no significant change in body weight of the treated marmosets during the treatment period.

### Reproductive Cycle

Before therapy 2 animals showed ovarian cycle activity, 5 showed irregular cycles and 1 no showed activity. After treatment 6 out of 8 animals showed ovarian cycle activity and 2 showed irregular cycles.

### Colour Doppler Scanning

Colour Doppler ultrasonic scanning was performed to measure blood flow in the reproductive tract (uterus and ovaries) area as well as in the bladder area. Decreased blood flow was found in all animals in both areas.

Individual blood flow measurements are provided in Table 1, 0=blood flow not visible, 1=weak blood flow, 2=medium blood flow and 3=massive blood flow. When comparing the median decrease in blood flow in all animals before and at the end of treatment, statistical significance (p<0.05) was achieved for both the bladder and reproductive tract, see Table 2.

TABLE 1

| Individual data on blood flow bladder | | | | | Individual data on blood flow repro tract | | | | |
|---|---|---|---|---|---|---|---|---|---|
| | Study week | | | | | Study week | | | |
| Animal | −2 (BL) | Week 1 | Week 4 | Week 6 | Animal | −2 (BL) | Week 1 | Week 4 | Week 6 |
| 1 | 2 | 2 | 1 | 1 | 1 | 2 | 2 | 1 | 1 |
| 2 | 3 | 3 | 2 | 1 | 2 | 2 | 2 | 1 | 1 |
| 3 | 1 | 1 | 1 | 1 | 3 | 1 | 1 | 0 | 0 |
| 4 | 1 | 1 | 1 | 1 | 4 | 1 | 1 | 1 | 0 |
| 5 | 3 | 3 | 3 | 2 | 5 | 3 | 3 | 2 | 2 |
| 6 | 2 | 2 | 2 | 1 | 6 | 2 | 2 | 1 | 1 |
| 7 | 2 | 2 | 1 | 1 | 7 | 2 | 2 | 2 | 2 |
| 8 | 3 | 3 | 2 | 1 | 8 | 3 | 2 | 2 | 2 |
| Median | 2 | 2 | 1.5 | 1 | Median | 2 | 2 | 1 | 1 |
| IQR | 1.75; 3 | 1.75; 3 | 1; 2 | 1; 1 | IQR | 1.75; 2.25 | 1.75; 2 | 1; 2 | 0.75; 2 |

TABLE 2

| Study Week | −2 (BL) | 6 (EoT) |
|---|---|---|
| Bladder | 2 (1.75; 3) | 1 (1; 1)* |
| Repro tract | 2 (1.75; 2.25) | 1 (0.75; 2)* |

Values are Median (25% quartile; 75% quartile);
*p < 0.05 compared with baseline (Wilcoxon Signed Rank Test)

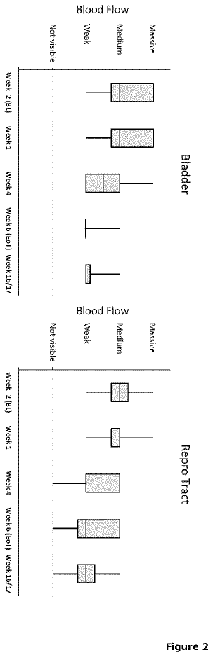

The changes in blood flow observed are shown in FIG. 2.

### Laparotomy

Laparotomy analysis was performed to assess endometriotic lesions. Pronounced reduction of endometric foci was found in all animals and all sizes of foci were affected by treatment.

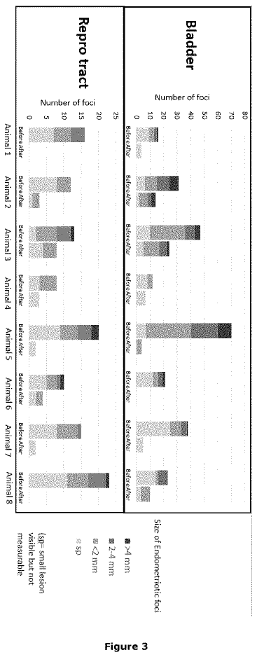

The changes in the individual number of endometriotic foci are shown in FIG. 3.

The individual data of endometriotic foci before and after treatment is shown in Table 3. When comparing the reduction in total number of endometriotic foci in all animals combined before and at the end of treatment, statistical significance (p<0.01) was achieved for both the bladder and reproductive tract, see table 4.

TABLE 3
Individual data on the number and size of endometriotic lesions on the bladder and reproductive tract
|Individual data on the number and size of endometriotic lesions on the bladder

| | Study week | | | | | | | | | |
|---|---|---|---|---|---|---|---|---|---|---|
| | Week −7/−8 (BL) | | | | | Week 7 (EoT) | | | | |
| | Lesion size | | | | | | | | | |
| Animal | s.p. | <2 mm | 2-4 mm | >4 mm | Total | s.p. | <2 mm | 2-4 mm | >4 mm | Total |
| 1 | 9 | 4 | 2 | 1 | 16 | 4 | 0 | 0 | 0 | 4 |
| 2 | 6 | 9 | 9 | 7 | 31 | 2 | 5 | 3 | 3 | 14 |
| 3 | 10 | 26 | 7 | 4 | 47 | 5 | 12 | 5 | 2 | 24 |
| 4 | 8 | 4 | 0 | 0 | 12 | 7 | 0 | 0 | 0 | 7 |
| 5 | 7 | 33 | 20 | 10 | 70 | 0 | 4 | 0 | 0 | 4 |
| 6 | 12 | 4 | 3 | 2 | 21 | 12 | 0 | 0 | 0 | 12 |
| 7 | 25 | 8 | 4 | 1 | 38 | 5 | 0 | 0 | 0 | 5 |
| 8 | 14 | 2 | 6 | 1 | 23 | 3 | 7 | 0 | 0 | 10 |
| Median | 9.5 | 6 | 5 | 1.5 | 27 | 4.5 | 2 | 0 | 0 | 8.5 |
| IQR | 7.75; 12.5 | 4; 13.25 | 2.75; 7.5 | 1; 4.75 | 19.75; 40.25 | 2.75; 5.5 | 0; 6.25 | 0; 0.75 | 0; 0.5 | 4.75; 12.5 |

Individual data on the number and size endometriotic lesions repro tract

| | Study week | | | | | | | | | |
|---|---|---|---|---|---|---|---|---|---|---|
| | Week −7/−8 (BL) | | | | | Week 7 (EoT) | | | | |
| | Lesion size | | | | | | | | | |
| Animal | s.p.* | <2 mm | 2-4 mm | >4 mm | Total | s.p. | <2 mm | 2-4 mm | >4 mm | Total |
| 1 | 7 | 5 | 4 | 0 | 16 | 0 | 0 | 0 | 0 | 0 |
| 2 | 8 | 4 | 0 | 0 | 12 | 1 | 2 | 0 | 0 | 3 |
| 3 | 2 | 6 | 4 | 1 | 13 | 4 | 4 | 0 | 0 | 8 |
| 4 | 3 | 5 | 0 | 0 | 8 | 3 | 0 | 0 | 0 | 3 |
| 5 | 9 | 5 | 4 | 2 | 20 | 2 | 0 | 0 | 0 | 2 |
| 6 | 5 | 3 | 1 | 1 | 10 | 2 | 2 | 0 | 0 | 4 |
| 7 | 8 | 6 | 1 | 0 | 15 | 2 | 0 | 0 | 0 | 2 |
| 8 | 11 | 6 | 5 | 1 | 23 | 0 | 0 | 0 | 0 | 0 |
| Median | 7.5 | 5 | 2.5 | 0.5 | 14 | 2 | 0 | 0 | 0 | 2.5 |
| IQR | 4.5; 8.25 | 4.75; 6 | 0.75; 4 | 0; 1 | 11.5; 17 | 0.75; 2.25 | 0; 2 | 0; 0 | 0; 0 | 1.5; 3.25 |

IQR: Interquartile range (25% quartile, 75% quartile)
*sp = visible but not measurable

TABLE 4

| | Bladder | | Repro tract | |
|---|---|---|---|---|
| | Study Week | | | |
| Lesion size | −2 (BL) | 7 (EoT) | −2 (BL) | 7 (EoT) |
| s.p. | 9.5 (7.75; 12.5) | 4.5 (2.75; 5.5) | 7.5 (4.5; 8.25) | 2 (0.75; 2.25) |
| <2 mm | 6 (4; 13.25) | 2 (0; 6.25) | 5 (4.75; 6) | 0 (0; 2) |
| 2-4 mm | 5 (2.75; 7.5) | 0 (0; 0.75) | 2.5 (0.75; 4) | 0 (0; 0) |
| >4 mm | 1.5 (1; 4.75) | 0 (0; 0.5) | 0.5 (0; 1) | 0 (0; 0) |
| Total | 27 (19.75; 40.25) | 6 (4; 12.5)** | 14 (11.5; 17) | 2.5 (1.5; 3.25)** |

Values are Median (25% quartile; 75% quartile);
**p < 0.01 compared with baseline (Wilcoxon Signed Rank Test)

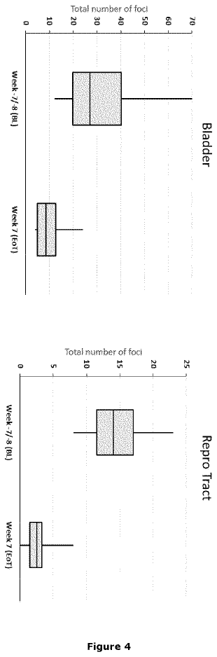

The changes in the number of endometriotic foci are shown in FIG. 4.

### Feeding Tree

The feeding tree was to test movement activity associated with pain, discomfort and/or distress. The feeding tree offers food at different positions to reach, which requires different levels of movement. If an animal has discomfort, distress or abdominal pain it will not search for food at upper areas or at all. Moreover, systematic search is reduced if memory is impaired. In the setting, each primate had to empty every reward-containing hideouts within 5 minutes to be fully successful. The response of the 8 animals was graded before, during and after treatment on a scale of 0-4 indicting the number of hideouts emptied within 5 minutes.

All animals improved their success rate in the feeding tree during treatment

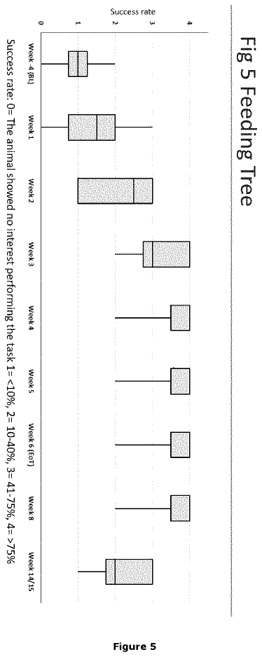

The changes in feeding tree behaviour over the course of the study are shown in FIG. 5, shown as median values.

### Wisconsin General Testing Apparatus

Memory and learning of the primates were tested. All animals are train-rewarded for the WGTA. The animal, which is sitting in the transport box, is either trained for special figures or places, which hide the reward. The observer can watch the behaviour of the animal, but the animal cannot see the observer.

The 8 animals were tested in the WGTA for memory by remembering either the figure or location of the hidden reward. Wellbeing of a primate is associated with willingness to search for food, which is also included by this test system. The animals have to find the reward underneath the three different figures three times within 5 minutes. Each animal is either trained for the location of the figure or figure itself.

During the treatment, all females showed increased success rate of correct figure or location where the reward was placed within time.

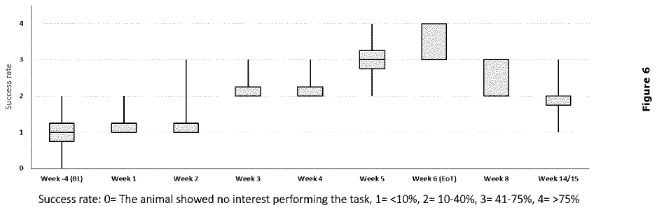

The changes in WGTA behaviour over the course of the study are shown in FIG. 6, shown as median values.

### Social Interaction

The documentation of grooming was carried out by daytime observation. These behaviour observations give information about wellbeing, while grooming is a marker for positive social activity. The social grooming was documented 1 h after breakfast and 1 h after lunch by observing the couples each time for 10 minutes. For each animal, the number of grooming interactions in the observed period was recorded.

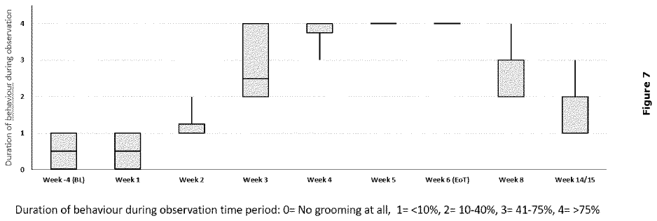

Grooming increased during treatment in all animals compared to before treatment. The changes in grooming behaviour over the course of the study are shown in FIG. 7, shown as median values.

1. A compound of formula I 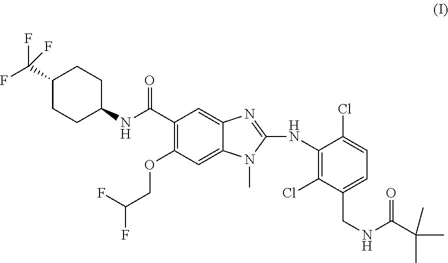 [SMILES: Cn1c(Nc2c(Cl)ccc(CNC(=O)C(C)(C)C)c2Cl)nc2cc(C(=O)N[C@H]3CC[C@H](C(F)(F)F)CC3)c(OCC(F)F)cc21]
or a pharmaceutically acceptable salt thereof, for use in the treatment or prophylaxis of endometriosis.

2. A method for the treatment or prophylaxis of endometriosis, comprising administering to a patient in need thereof a therapeutically effective amount of a compound of formula I as defined in claim 1, or a pharmaceutically acceptable salt thereof.

3. The use of a compound of formula I as defined in claim 1, or a pharmaceutically acceptable salt thereof, in the manufacture of a medicament for the treatment or prophylaxis of endometriosis.

4. A pharmaceutical formulation comprising a compound of formula I as defined in claim 1, or a pharmaceutically acceptable salt thereof, and optionally one or more pharmaceutically acceptable excipients, for use in the treatment or prophylaxis of endometriosis.

5. A combination product comprising:
(I) a compound of formula I as defined in claim 1, or a pharmaceutically acceptable salt thereof; and
(II) one or more other therapeutic agent useful in the treatment or prophylaxis of endometriosis,
wherein each of components (I) and (II) is formulated in admixture, optionally with one or more a pharmaceutically-acceptable excipient.

6. A kit-of-parts comprising:
(a) a pharmaceutical formulation as defined in claim 4; and
(b) a pharmaceutical formulation comprising one or more other therapeutic agent useful in the treatment or prophylaxis of endometriosis, optionally in admixture with one or more pharmaceutically-acceptable excipients,
wherein components (a) and (b) are each provided in a form that is suitable for administration in conjunction with the other.
